(19) United States
(12) Patent Application Publication
(10) Pub. No.: US 2020/0306060 A1
(43) Pub. Date: Oct. 1, 2020

(54) SYSTEM CONSISTING OF AT LEAST ONE ORTHOPAEDIC COMPONENT, AND AN OPERATOR CONTROL AND/OR FEEDBACK DEVICE

(71) Applicant: Otto Bock Healthcare Products GmbH, Wien (AT)

(72) Inventors: Alexander PAPPE, (US); Andreas WEIGL-POLLACK, (US); Erik ALBRECHT-LAATSCH, (US); Michael NOLTE, (US); Robert HOFFMANN, (US); Robert KAITAN, (US); Luis SAGMEISTER, (US); Thomas PAUSER, (US); Andreas SCHRAMEL, (US)

(73) Assignee: Otto Bock Healthcare Products GmbH, Wien (AT)

(21) Appl. No.: 15/733,256

(22) PCT Filed: Dec. 18, 2018
(86) PCT No.: PCT/EP2018/085656

§ 371 Date: Jun. 17, 2020

(30) Foreign Application Priority Data
Dec. 22, 2017 (DE) 10 2017 131 196.3

(51) Int. Cl. A61F 2/70

(52) U.S. Cl. CPC A61F 2/70; A61F 2002/6809

(57) ABSTRACT

A system consisting of at least one orthopaedic component, having at least one stored energy source and multiple electrical and/or electronic devices, and at least one operator control device and/or feedback device, which is assigned to the electrical and/or electronic devices and is coupled to them.

The invention relates to a system made of at least one orthopedic component comprising at least one energy store and a plurality of electrical and/or electronic devices.

Orthopedic components include orthoses, prostheses, wheelchairs, data loggers, radio modules, accumulators and parts of orthoses, prostheses and wheelchairs, for example prosthetic or orthotic knee joints, prosthetic feet, tube adapters, prosthetic hands, prosthetic or orthotic elbow joints, rotary adapters, prosthetic shafts, splints and other joints, and fastening devices for securing orthoses or prostheses to a patient.

At least one electrical and/or electronic device can be provided on such an orthopedic component, for example for recording data, reproducing data or transmitting data. The electrical and/or electronic device can be used to set a damper or said device can be provided and configured to realize a drive to bring about displacements or pivoting of components with respect to one another or to process data, for example in the form of a microprocessor. In order to be able to supply these electrical and/or electronic devices with power when necessary, at least one energy store is assigned to the at least one orthopedic component so that data can be stored or read, a radio connection can be established, drives can be supplied with power or other actions can be carried out.

Monitoring of the electrical and/or electronic devices may be difficult, particularly in the case of a system made of a plurality of orthopedic components, for example a prosthetic knee joint and a prosthetic ankle joint with further components, which for example have sensors or which are embodied as actuation devices for activating, deactivating or changing the resistances or drives in the respective prosthetic joints, disposed therebetween.

It is an object of the present invention to provide a system that allows the at least one orthopedic component comprising a plurality of electrical and/or electronic devices to be operated more comfortably.

According to the invention, this object is achieved by a system having the features of the main claim. Advantageous embodiments and developments of the invention are described in the dependent claims, the description and the figures.

The system made of at least one orthopedic component comprising at least one energy store and a plurality of electrical and/or electronic devices provides for at least one operator control device and/or feedback device to be provided, which is assigned to the electrical and/or electronic devices and coupled thereto. The operator control and/or feedback device allows monitoring and driving or setting of all electrical and/or electronic devices. Thus, for example, there could be a charge state display of all energy stores or functionality confirmation of all electrical and/or electronic devices by the feedback device. Likewise, all electrical and/or electronic devices could be activated or deactivated or could be switched on or switched over in respect of the mode of operation thereof by way of a single operator control device.

The electrical and/or electronic device can be embodied as a drive, a switch, a solenoid, a data processing device and/or a sensor. In particular, an electromotive drive is provided as the drive; in principle, linear drives can also be provided in addition to rotary drives. Different switching states, e.g., for valves in a hydraulic or pneumatic circuit, can be switched or changed in terms of their position by means of a solenoid. In addition to active elements, which bring about a change within the system, sensors can also be used as electrical and/or electronic devices in order to capture forces, acceleration, angular positions of components with respect to one another, moments, speeds, relative spatial positions or else brightness levels or ambient volume levels. Likewise, movement cycles or load cycles can be captured by way of the sensors; these are then transmitted to the operator control and/or feedback device such that there can be a simple evaluation in a central operator control and/or feedback device.

The operator control and/or feedback device is preferably coupled to the at least one energy store in order to bring about or lift a separation between the electrical and/or electronic devices and the energy store. The coupling to the energy store moreover makes it possible for the feedback device to bring about an active signal output, for example an optical signal output, an acoustic signal output or the like, in order to inform the user, for example the patient or an orthopedic technician, about the current state, the performed load cycles or possible future settings. To this end, the feedback device has at least one feedback element, which is embodied as an optical, tactile, acoustic and/or thermal output device. This allows the necessary or requested information to be provided by the feedback device in different ways. In addition to purely optical display, for example on a display, an acoustic output device in the form of a loudspeaker can be used either to output a voice message or, by way of a signal tone or sequence of signal tones, to indicate that the orthopedic component is activated or deactivated or indicate the current mode thereof. Such information output can be assisted by a tactile output device, for example a device for generating vibrations. The tactile output device can also be embodied as the only feedback element. A further information channel can be provided for the user by way of a thermal output device.

The at least one operator control device and/or feedback device is reversibly attached to the orthopedic component in one development of the invention, for example in form-fit fashion by way of screws, hook-and-loop fasteners, clips, hooks, rotary fasteners, belts, straps or tabs, into which the operator control and/or feedback device is inserted or partially inserted. Reversible force-fit coupling or fastening can be achieved by way of magnetic coupling, in particular. Reversible fastening can also be implemented by way of a combination of form-fit and force-fit.

The operator control device can be embodied as a heat sensor, magnetic field sensor, capacitive sensor, optical sensor, proximity sensor, radar-assisted motion sensor, piezo element and/or simple electrical switch or contact in order to allow, in a number of ways, the orthopedic component to be easily and reliably operated. By way of example, using a radar-assisted motion sensor, the orthopedic component or the orthopedic components of the system could be operated by way of a hand gesture control. Likewise, an optical sensor with proximity detection can be used to ascertain whether operator control should occur and how the manner of the movement should be interpreted. An electrical signal can be triggered by pressing a piezo element. A heat sensor can detect a difference in heat, for example if a finger or another part of the body is placed on the corresponding sensor in order to output an input signal to a control device. The same applies to magnetic field sensors or capacitive sensors, and so the orthopedic component can be set, activated or deactivated via a touchscreen as an operator control element.

In addition to the representation of the respectively set function and the charge state of the energy stores or the energy store, the feedback element can also be used for automatic brightness and/or volume control. To this end, a brightness sensor and/or a microphone are provided on or in the feedback device, via which the ambient brightness or the ambient volume level are measured. Backlighting is then activated or deactivated or the acoustic output is adapted to the ambient volume level via an adaptive brightness and/or volume control.

The operator control and/or feedback device can have form-fit elements and/or force-fit elements for securing to the orthopedic component. This allows the operator control and/or feedback device to be positioned freely on the orthopedic device, for example the orthosis, prosthesis or wheelchair, and be secured there, if need be in detachable fashion. Clips, hook-and-loop fasteners, hooks or threaded elements could be provided as form-fit elements, which enable a permanent, yet detachable connection to the orthopedic component. The operator control and/or feedback device can be secured by force fit on the orthopedic component using magnets.

Exemplary embodiments of the invention will be explained in more detail below with reference to the attached figures. In the drawing:

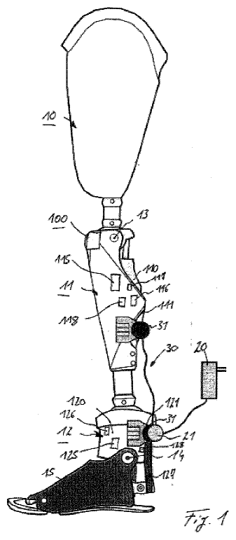

FIG. 1—shows a first variant of the system; and

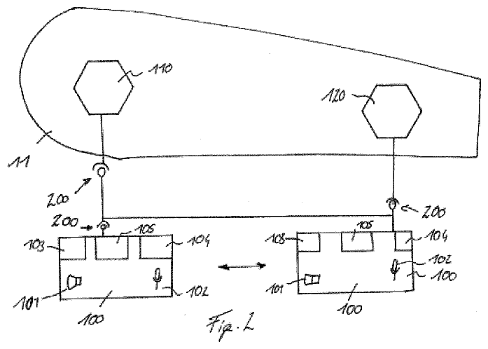

FIG. 2—shows a second variant.

In a schematic illustration, FIG. 1 illustrates a prosthesis of a lower extremity with a plurality of orthopedic components 10, 11, 12, 15. A thigh shaft with a connection adapter for fastening to an upper part of a prosthetic knee joint is shown as first orthopedic component 10. A second orthopedic component 11 in the form of a lower leg part is disposed on the prosthetic knee joint so as to be pivotable about a pivot axis 13. A third orthopedic component 12 in the form of a prosthetic ankle joint is fastened to the lower leg part as a second orthopedic component 11, a prosthetic foot 15 as a fourth orthopedic component being mounted to said prosthetic ankle joint so as to be pivotable about a pivot axis 14. The thigh shaft as first orthopedic component 10 is a purely passive component and is used to hold a thigh stump in order to be able to securely fasten the prosthetic leg to the patient. The prosthetic leg can be mechanically secured to the patient via the thigh shaft and a prosthesis liner disposed therein, for example by way of a negative pressure system. An alternative to a multi-part prosthesis, the system could also be embodied as an orthosis; likewise, a wheelchair or a prosthetic or orthotic device for an upper extremity, for example a prosthetic arm, an arm orthosis or shoulder orthosis, can be used as an orthopedic component. Combinations of prostheses, orthoses and wheelchairs or other mobility aids are also considered as a system.

In the illustrated system, a first electrical and/or electronic device 110 in the form of a hydraulic actuator is disposed in the second orthopedic component 11. The hydraulic actuator can be embodied as a passive component and can have a hydraulic damper which has positioners used to open or close valves so as to be able to set an extension resistance and/or a flexion resistance. Moreover, a store 115 for storing electrical energy can be disposed on or assigned to the hydraulic actuator in order to supply the positioners with energy. In an embodiment of the electrical and/or electronic device 110 as an active actuator, a pump device or a mechanical energy store, e.g., a spring or a pressure accumulator, is assigned to the hydraulic damper, by means of which it is possible to effect or assist a movement of the thigh shaft relative to the lower leg. To this end, energy from the energy store 115 is converted into kinetic energy such that, for example, a piston rod is moved out of the hydraulic actuator in order to assist or effect an extension movement. Conversely, hydraulic fluid can be pumped around by a pump or a pressure accumulator such that a piston rod is retracted into a housing of the hydraulic actuator in order to shorten the distance between two fastening points of the hydraulic actuator on the upper part and the lower part of the prosthesis joint so as to carry out a flexion movement.

Distally to the first electrical and/or electronic component 110, the second electrical and/or electronic component 120 is disposed in the orthopedic component 12 in the form of a prosthetic ankle joint. An electrical and/or hydraulic actuator, which can be supplied with electrical energy via a store 125 for electrical energy, can likewise be disposed within the prosthetic ankle joint. In addition to the store 125 for electrical energy, a control device 126 is disposed in the prosthetic ankle joint, disposed on the control device 116 in the first electrical and/or electronic device 110, for example in order to activate and deactivate a drive 118, 128 or to process data of a sensor 117, 127, to store sensor data and to use said sensor data further for control purposes. A data memory and a processing circuit can also be integrated in the control device 116, 126.

The first orthopedic component 11 and the second orthopedic component 12 each have a supply connector 111, 121, by means of which energy and/or data can be supplied to the respective electrical and/or electronic device 110, 120. The data and the electrical energy can be transferred from a charging station 20 to the respective supply connector 111, 121. To this end, a plug 21 that is compatible with the respective supply connector 111, 121, is disposed on the charging station 20. In the illustrated exemplary embodiment of FIG. 1, the plug 21 of the charging station 20 is coupled to a cascading plug system of a plug connection 30, which connects both supply connectors 111, 121 to one another. Two plugs 31 are disposed on the plug connection 30, said plugs having contacts on one side which have an embodiment compatible with the contacts of the respective supply connectors 111, 121. On the side distant from the supply connectors 111, 121, the plugs 31 have receptacles or sockets that are compatible with the contacts of the plug 21 of the charging station 20.

If the plug 21 of the charging station 20 is plugged onto the back side of a plug 31 which is connected via a cable to a corresponding plug 31 such that both supply connectors 111, 121 are interconnected, it is possible to transfer energy and data from the charging station 20 to both the first and second electronic and/or electrical device 110, 120. As a result, the stores 115, 125 for storing electrical energy are filled and the control devices 116, 126 are supplied with data such as programs, control data, software updates or the like. By way of the system made up of charging station 20 with plug 21, supply connectors 111, 121 and plug connection 30 with the plugs 31, it is possible to provide an electrical connection system for orthopedic components 11, 12, in particular for orthoses, prostheses and/or wheelchairs, by means of which it is possible to charge the respective electrical and/or electronic devices 110, 120 and to interconnect these in order to distribute energy from the respective energy stores 115, 125 among one another or coordinate control procedures with one another. By way of the system, it is possible not only to supply external data from the charging station 20 and electrical energy to the orthopedic system, for example the orthosis, prosthesis or the wheelchair, but also to facilitate an energy and/or data interchange between the respective orthopedic components 11, 12 within the orthopedic device.

Should there be different maximum powers required in the respective consumer, for example the drive 118, 128, in the different components 11, 12 with the different electrical and/or electronic devices 110, 120, the respective maximum power of a consumer 118, 128 or of a control device 116, 126 required could be encoded by way of an electrical resistor. As a result of the compatibility of the supply connectors 111, 121 with the respective plugs 21, 31, it is possible to provide a plurality of charge connectors on an orthopedic component in order to make it easier for a user to couple the orthopedic auxiliary means to a charge station 20. As a result, the user can freely select the supply connector 111, 121 most easily reached by them, and so one or more connectors are available for centrally charging all electrical and/or electronic devices 110, 120. Charging with electrical energy and supplying with data can be implemented simultaneously or sequentially. For example, if the electrical and/or electronic devices 110, 120 are not coupled to one another via the plug connection 30, data and energy can be transmitted in succession via the charging station 20 through the plug 21. The respective electronic and/or electrical device 110, 120 is provided with a code such that the device currently intended to be supplied with energy and/or data can be identified by the charging station 20, and so both the correct amount of energy and the correct data are transmitted.

If the charging station 20 is not connected, there can be data and/or energy interchange between the devices 110, 120 by the plug connection 30. It is also possible to combine a plurality of orthopedic auxiliary means via a plug connection 30, for example an orthosis or prosthesis with a wheelchair which, for example, has a greater energy store in the form of a battery such that, for the purposes of maintaining the mobility of the user or the patient, energy can be transferred from the wheelchair to the orthosis or prosthesis.

In the illustrated exemplary embodiment as per FIG. 1, an operator control and/or feedback device 100 is disposed on the lower leg part and reversibly fastened to said lower leg part 11, for example by force-fit elements of form-fit elements, by means of which it is possible to secure the operator control and/or feedback device 100 to the orthopedic component 11 and remove it therefrom again. The form-fit elements can be designed as plugs, clips, hook-and-loop fasteners, clamps, plug-in elements or else screws, and magnets are particularly suitable as force-fit elements, by means of which simple force-controlled securing and removal of the operator control and/or feedback device 100 can be implemented. Force-fit coupling is advantageous in that, as a rule, there can be damage-free separation of the operator control and/or feedback device 100 from the respective orthopedic component should the user of the prosthesis, the orthosis or the wheelchair hit an object. Moreover, virtually any positioning is possible at the point appearing most suitable to the user, provided sufficient magnetic or ferromagnetic elements are present on the orthopedic device. By way of the securing region, for example by way of plugs or contact elements present both on the operator control and/or feedback device 100 and at the fastening position on the respective orthopedic component 10, 11, 12, 15, it is also possible to implement energy- and data-transmitting coupling between the orthopedic component with the disposed or assigned energy store 115, 125 and the respective electrical and/or electronic device 110 with the drives, switches, data processing devices and sensors.

For the operator control part, the operator control and/or feedback device 100 can have a heat sensor, magnetic field sensor and capacitive sensor, optical sensor, proximity sensor, radar-assisted motion sensor, a piezo element and/or an electrical contact in order to easily allow a user of the orthopedic component or of the system made of an orthopedic component and the operator control and/or feedback device to influence the electrical and/or electronic devices with the components, devices or apparatuses disposed thereon or coupled therewith, in particular to switch these on, switch these off or undertake changes in their settings. In the case of a combined configuration with a feedback device, the feedback device can have at least one feedback element embodied as an optical, tactile, acoustic or thermal output device. By way of an optical output device, the user of the orthopedic component is informed, by means of a display, about the state of the orthopedic component or the electrical and/or electronic device, for example what resistances are present in a damping unit, what the charge state of an energy store is, how amounts of energy are distributed in a plurality of energy stores, what software version is present, whether a date for servicing has to be set or the like. A tactile output device can output, for example, a vibration signal that indicates an activation or deactivation of an electrical and/or electronic component. By means of an acoustic output, for example by means of a signal tone or a voice output, it is possible to communicate to the user the mode in which the orthopedic component is in or the state in which an energy store device is.

The operator control and/or feedback device 100 can have a brightness sensor, for example in order to automatically illuminate an operator control field or illuminate or dim devices on the orthopedic component. In any case, a microphone can be coupled to the operator control and/or feedback device 100, or embodied therein, for example to undertake adaptive volume control during an output with a loudspeaker or else to input acoustic commands by the microphone using a voice input.

FIG. 2 shows a schematic illustration of an orthopedic component 11 as per FIG. 1 in the form of a lower leg part with a first electrical and/or electronic device 110 as a hydraulic actuator for influencing a flexion movement and extension movement between a thigh shaft and a lower leg part, and with a second electrical and/or electronic device 120 in the form of a prosthetic ankle joint. Coupling devices in the form of form-fit elements and/or force-fit elements 200 are disposed on the electrical and/or electronic devices 110, 120, said coupling devices possibly also having devices for electrical coupling and data processing coupling at the same time. As an alternative to mechanical coupling by way of the form-fit elements 200, which could also be embodied as lockable plugs, for example, or by way of force-fit elements 200 with associated contact surfaces for energy and data transmission, the data transmission, in particular, could also be implemented wirelessly. The operator control and/or feedback device 100 can have a loudspeaker 101 and a microphone 102 in order to be able to carry out acoustic output as a feedback function and acoustic input via voice control during an operator control function. Likewise, a tactile output device 103 in the form of a vibration field and/or a display 105, and a tactile input device or a heat sensor, a magnetic field sensor, an optical sensor or a proximity sensor, or else an electrical contact 104, could be present in order to be able to enter, via the operator control and/or feedback device 100, corresponding input commands into the orthopedic component 11 for the purposes of controlling electrical and/or electronic device 110. Both electrical and/or electronic devices 110, 120 can be driven and feedback signals from the latter can be received by way of the one operator control and/or feedback device 100. It is likewise possible to provide a second operator control and/or feedback device 100, which is assigned to the second electrical and/or electronic device 120. Here, too, the coupling can be implemented by way of form-fit or force-fit elements 200 with integrated electrical and data-transmitting coupling or else, possibly, without data-transmitting coupling. The two operator control and/or feedback devices 100 can be synchronized wirelessly with one another such that all electrical and/or electronic devices 110, 120 can be actuated and data or feedback can be received therefrom by each operator control and/or feedback device 100. In FIG. 2, the two operator control and/or feedback devices 100 are reversibly coupled to the electrical and/or electronic devices 110, 120 by the force-fit and/or form-fit elements 200. The right-hand operator control and/or feedback device 100 is embodied to be reversibly fastened to both force-fit and/or form-fit elements 200 in order to operate both electrical and/or electronic devices 110, 120 or be able to receive feedback therefrom and output said feedback. Provision is also made for an operator control and/or feedback device 100 to be used for each electrical and/or electronic device 110, 120. In the illustrated combination, the connection of the left-hand operator control and/or feedback device 100 can bring about an assignment to the left-hand electrical and/or electronic device 110; in that case, the right-hand operator control and/or feedback device 100 is only responsible for the right-hand electrical and/or electronic device 120. In one variant, both operator control and/or feedback devices 100 can also communicate with both electrical and/or electronic devices 110, 120.

1. A system comprising:
at least one orthopedic component comprising:
at least one energy store;
a plurality of electronic devices;
at least one operator control device or feedback device, which is assigned to the plurality of electronic devices and coupled thereto.

2. The system as claimed in claim 1, wherein the each of the plurality of electronic devices includes at least one of a drive, a switch, a solenoid, a data processing device, and a sensor.

3. The system as claimed in claim 1, wherein the at least one operator control or feedback device is coupled to the at least one energy store.

4. The system as claimed in claim 1, wherein the at least one operator control device or feedback device is reversibly attached to the at least one orthopedic component.

5. The system as claimed in claim 1, wherein the at least one operator control device has at least one of a heat sensor, magnetic field sensor, capacitive sensor, optical sensor, proximity sensor, radar-assisted motion sensor, piezo element, and electrical contact.

6. The system as claimed in claim 1, wherein the at least one feedback device has at least one feedback element, which is embodied as at least one of an optical, tactile, acoustic, and thermal output device.

7. The system as claimed in claim 1, wherein the at least one operator control or feedback device has at least one of a brightness sensor and a microphone and at least one of adaptive brightness and volume control.

8. The system as claimed in claim 1, wherein the at least one operator control or feedback device has at least one of form-fit elements and force-fit elements for securing to the at least one orthopedic component.

9. A system comprising:
at least one orthopedic component comprising:
at least one energy store;
a plurality of electronic devices;
at least one of an operator control device and a feedback device, which is physically and electrically coupled to the plurality of electronic devices.

10. The system as claimed in claim 9, wherein the electronic device comprises at least one of a drive, a switch, a solenoid, a data processing device, and a sensor.

11. The system as claimed in claim 9, wherein the at least one of the operator control device and the feedback device is coupled to the at least one energy store.

12. The system as claimed in claim 9, wherein the at least one of the operator control device and the feedback device is reversibly attached to the at least one orthopedic component.

13. The system as claimed in claim 9, wherein the operator control device comprises at least one of a heat sensor, magnetic field sensor, capacitive sensor, optical sensor, proximity sensor, radar-assisted motion sensor, piezo element, and electrical contact.

14. The system as claimed in claim 9, wherein the feedback device has at least one feedback element, which is embodied as at least one of an optical, tactile, acoustic, and thermal output device.

15. The system as claimed in claim 9, wherein the at least one of the operator control device and the feedback device comprises at least one of a brightness sensor and a microphone and at least one of an adaptive brightness and a volume control.

16. The system as claimed in claim 9, wherein the at least one of the operator control device and the feedback device comprises at least one of form-fit elements and force-fit elements for securing to the at least one orthopedic component.
